(19) United States
(12) Patent Application Publication
(10) Pub. No.: US 2024/0065822 A1
(43) Pub. Date: Feb. 29, 2024

(54) NOVEL DEVICE FOR TREATMENT OF AORTIC DISSECTIONS

(71) Applicant: Ashish Mitra, Sydney (AU)

(72) Inventor: Ashish Mitra, Sydney (AU)

(21) Appl. No.: 18/261,093

(22) PCT Filed: Jan. 13, 2022
(86) PCT No.: PCT/AU2022/050012

§ 371 Date: Jul. 11, 2023

Related U.S. Application Data
(60) Provisional application No. 63/136,644, filed on Jan. 13, 2021.

(51) Int. Cl. A61F 2/07; A61B 17/12; A61F 2/958

(52) U.S. Cl. CPC A61F 2/07; A61B 17/12031; A61B 17/12118; A61B 17/12172; A61F 2/958; A61F 2002/828; A61F 2230/0013

(57) ABSTRACT

A system and a method for treating an aortic dissection comprising deploying a crescent-shaped stent graft in the false lumen through the re-entry point. The stent graft comprises a graft structure secured to a stent structure. The stent graft is geometrically configured to block or occlude the blood flow through to the false lumen.

### CROSS-REFERENCE TO RELATED PATENT DOCUMENTS

This patent application claims the benefit of priority of U.S. Provisional Patent Application No. 63/136,644, titled “NOVEL DEVICE FOR TREATMENT OF AORTIC DISSECTIONS”, filed on Jan. 13, 2021, which is hereby incorporated by reference in its entirety.

### TECHNICAL FIELD OF INVENTION

The present invention generally relates to a system and method for treatment of aortic dissection and more particularly, it pertains to a medical implant that functionally occludes false lumen formed between layers of aorta.

### BACKGROUND

An aortic dissection is a dangerous condition with a high mortality rate. In an aortic dissection, a tear typically develops in the intima of the aorta that propagates along the vessel wall separating the inner layer of the aorta from the outer layer. Blood enters the space between the layers creating a false lumen. Several additional tears or entry points can be created between true lumen of the aorta and the false lumen. In the acute phase, dissections may close off perfusion from the aorta to vital organs. In the chronic phase, the weakened tissue can develop into aneurysm and ultimately rupture. According to the Stanford classification, dissections involving the ascending aorta are referred to as type A dissections and dissections involving only the descending aorta are referred to as type B dissections. Current treatments for dissections include medical management to lower the blood pressure of the patient and reduce the hemodynamic stresses on the diseased vessel. If dissections are symptomatic, surgical intervention is necessary. Portions of the diseased aorta are replaced by a surgical graft and the dissection flap is reattached. More recently, endovascular stent grafts have been used to close the primary entry point into the false lumen with the goal to thrombose the false lumen and maintain patency of the true lumen. Often only the primary entry point of a dissection is covered by the stent graft allowing continuous pressurization of the false lumen through secondary entry points. Long term, a pressurized false lumen tends to expand and could lead to aneurysm. The presence of only partial thrombosis with comparison to a fully thrombosed or patent false lumen increases the risk of death by a factor of 2.7 (Tsai et al. N Engl J Med 2007; 357:349-59). Even with endovascular intervention, there is evidence to suggest that treatment is not as effective to completely thrombose the false lumen as it should be (Sayer et al. Eur J Vasc Endovasc Surg 2008; 36(5):522-9). Further, it appears that endovascular stent graft treatment is not an effective treatment for chronic type B dissection patients, with just 36% of patients developing a fully thrombosed false lumen 2 years post-op.

There is ample clinical data suggesting that complete occlusion/thrombosis of the false lumen is not occurring in a large number of patients undergoing treatment for acute and chronic type A and type B aortic dissections. This is of particular concern, given the evidence supporting the importance of complete false lumen occlusion/thrombosis. There is a clear need for an improved method to treat aortic dissections. The current application provides novel solutions to the treatment of aortic dissections.

In order to achieve a fully thrombosed false lumen, numerous cases of false lumen embolization and/or occlusion techniques have been documented, with limited success. All share the common goal of initiating thrombosis by insertion of an embolization device (coils, plug or stent graft) into the false lumen, most frequently via the secondary entry tears [the primary entry tears are frequently covered by an endovascular stent graft].

In a review by Hofferbeth et al. (J Thorac Cardiovasc Surg 2014; 147(4)21240-5), 10 patients undergoing embolization in order to treat continued false lumen perfusion after using endovascular stent grafting only 20% patients had full false lumen thrombosis in both the thoracic and abdominal aorta (using coils and balloon occlusion), while the rest had at least some partial thrombosis. In another study by Idrees et al. (J Vasc Surg 2014; 60(6):1507-13) where primarily covered stent plugs/occluders were inserted into the false lumen, 71% of the patients had a fully thrombosed false lumen—a better result than Hofferbeth et al., but still markedly suboptimal.

While the aforementioned literature points to embolization as a possible approach to cause complete false lumen thrombosis, it has the following key limitations: (a) limited clinical efficacy—this can be attributed to the geometrically inadequate conformation achieved by such devices, which leads to persistent leak sites allowing perfusion of the false lumen through the re-entry tear—the shape of the false lumen in that of a crescent (in cross-section) and it is geometrically not possible to completely fill this space/cross-section with the currently available embolization devices—there will always be some space left unoccluded (b) limited commercial adoption/usage of the approach as: (i) the coils, plugs, etc. are not indicated for occluding a large volume/space such as a re-entry tear of an aortic dissection, and therefore there is no existing direction on methods of use in such an application (ii) it is extremely difficult to establish standard procedures/instructions for use for such devices relating to treatment of an aortic dissection as they are not inherently designed for this application (are used as “make do” devices).

An additional approach, as discussed in prior art U.S. Pat. No. 9,393,100B2, is to fill the false lumen with an inflatable bag. This approach has similar limitations to the embolization devices, where geometrically there will be a non-conformance of the bag to the false lumen thereby limiting the clinical efficacy.

The cross-section of the false lumen has a substantially crescent shape. The existing devices and techniques are unable to completely occlude crescent shaped false lumen owing to its geometry. Thus, there is a need for an implant that can completely occlude the re-entry tear of such shape by addressing its geometry.

### BRIEF SUMMARY

Before the present systems and methods, enablement are described, it is to be understood that this application is not limited to the particular systems, and methodologies described, as there can be multiple possible embodiments which are not expressly illustrated in the present disclosures. It is also to be understood that the terminology used in the description is for the purpose of describing the particular versions or embodiments only and is not intended to limit the scope of the present application.

It is an object of the present invention to provide a stent graft or implant that functionally fully occludes the false lumen due to its geometric shape and constructional features.

It is an object of the present invention to provide a stent graft that has substantially same shape as of cross-section of the false lumen.

It is an object of the present invention to provide a stent graft that enables complete thrombosis in the false lumen by blocking blood flow through to the false lumen.

It is an object of the present invention to provide a balloon that enables the said stent graft to functionally fit better in the anatomy of the false lumen.

Embodiments of the present invention disclose a system for treating an aortic dissection, the system comprising a stent graft for deploying in a false lumen. The said stent graft comprises: a graft structure comprising an inner graft and an outer graft. The inner graft has a top edge and a bottom edge, and the outer graft has a top edge and a bottom edge. The said stent graft further comprises a stent structure secured to the graft structure. The stent structure comprises at least one inner stent and at least one outer stent connected to the graft structure, wherein at least one inner stent is connected to the inner graft and at least one outer stent is connected to the outer graft. Either the top edge of the inner graft and the top edge of the outer graft are joined together to close the top edge of the stent graft, or the bottom edge of the inner graft and the bottom edge of the outer graft are joined together to close the bottom edge of the stent graft, or both the top and bottom edges of the stent graft are closed in the manner described. The said stent graft is configured to block the blood flow through the false lumen.

In an embodiment, the system comprises a balloon for enabling the said stent graft to functionally fit better in the anatomy of the false lumen.

In an embodiment, the system comprises a catheter for endovascularly deploying the said stent graft in the false lumen.

In an embodiment, the inner graft has a larger radius of curvature than the outer graft.

In an embodiment, the inner graft, the outer graft, the inner stents and the outer stents are connected to define the overall crescent shape of the said stent graft.

In an embodiment, a hole is provided at the top edge of the said stent graft.

In an embodiment, the hole is provided at the top and bottom edges of the said stent graft where the said stent graft has closed both top and bottom edges.

In an embodiment, the balloon has a cutting or ablating arrangement at the tip of the balloon to make a hole at the top edge of the said stent graft in situ.

In an embodiment, the system comprises a true lumen stent graft for deploying in a true lumen wherein the true lumen stent graft permits blood flow through the true lumen. The true lumen stent graft is substantially tubular in shape and circular in cross section and is generally known in the conventional art for the treatment of aortic dissections.

Embodiments of the present invention disclose a method for treating an aortic dissection. The method comprising steps of providing a stent graft wherein the said stent graft comprises: a graft structure comprising an inner graft and an outer graft; wherein the inner graft has a top edge and a bottom edge; and the outer graft has a top edge and a bottom edge; a stent structure secured to the graft structure; wherein the stent structure comprises at least one inner stent and at least one outer stent connected to the graft structure; wherein at least one inner stent is connected to the inner graft and at least one outer stent is connected to the outer graft; wherein the top edge of the inner graft and the top edge of the outer graft are joined together to close the top edge of the said stent graft; deploying the said stent graft in the false lumen of an aortic dissection at/around re-entry point; deploying a true lumen stent graft in a true lumen of an aortic dissection; wherein the said stent graft deployed in the false lumen blocks blood flow from flowing through the false lumen and the true lumen stent graft permits blood flow from flowing through the true lumen.

In an embodiment, the method comprises making a hole at the top edge of the said stent graft in situ.

In an embodiment, the method comprises making a hole at the top edge of the said stent graft by using the cutting or ablating arrangement of the balloon.

In an embodiment, the method comprises endovascularly deploying the said stent graft in the false lumen by using a catheter.

In an embodiment, the method comprises deploying a balloon to enable the said stent graft to functionally fit better in the anatomy of the false lumen.

In an embodiment, the inner graft, the outer graft, the inner stents and the outer stents are connected to define the overall crescent shape of the said stent graft.

In an embodiment, the bottom edge of the inner graft and the bottom edge of the outer graft are joined together to close the bottom edge of the said stent graft.

In an embodiment, the top and bottom edges of the inner graft and the top and bottom edges of the outer graft are respectively joined together to close the top and bottom edges of the said stent graft.

Various objects, features, aspects and advantages of the present invention will become more apparent from the following detailed description of the embodiments of the invention, along with the accompanying drawings in which like numerals represent like components.

### BRIEF DESCRIPTION OF THE DRAWINGS

The foregoing summary, as well as the following detailed description of preferred embodiments, is better understood when read in conjunction with the appended drawings. There is shown in the drawings example embodiments, however, the application is not limited to the specific system and method disclosed in the drawings.

FIG. 1 illustrates a non-limiting example of an aortic dissection.

FIG. 2 illustrates a cross sectional view of the aortic dissection of FIG. 1.

FIGS. 3-5 illustrates a perspective view, a top view and a front view of a stent graft or proposed implant respectively, according to an embodiment of the present invention.

FIG. 6 illustrates a perspective view of a stent graft or implant comprising an inner stent and an outer stent, according to another embodiment of the present invention.

FIG. 7 illustrates a perspective view of a stent graft or implant comprising four inner stents and four outer stents, according to yet another embodiment of the present invention.

FIG. 8 illustrates a perspective view of a stent graft, according to another embodiment of the present invention.

FIG. 9 illustrates the stent graft of FIGS. 3-5 in deployed position at re-entry point of the false lumen.

FIG. 10 illustrates a perspective view of a stent graft having a closed top edge as well as a closed bottom edge, according to another embodiment of the present invention.

FIG. 11 illustrates the stent graft of FIG. 10 in deployed position at re-entry point of the false lumen.

FIG. 12 illustrates a perspective view of a stent graft having a hole at top edge, according to yet another embodiment of the present invention.

FIG. 13 illustrates a perspective view of a catheter with a balloon for enabling the stent graft to functionally fit better in the anatomy of the false lumen, according to an embodiment of the present invention.

FIG. 14 illustrates a stent graft having a base diameter and an overall height.

FIG. 15 illustrates a graph showing pressure reading in true lumen and false lumen wherein the false lumen does not include the stent graft.

FIG. 16 illustrates a graph showing pressure reading in true lumen and false lumen wherein a stent graft is deployed in the false lumen.

### DETAILED DESCRIPTION

Some embodiments, illustrating its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any methods, and systems similar or equivalent to those described herein can be used in the practice or testing of embodiments, the preferred methods, and systems are now described. The disclosed embodiments are merely exemplary.

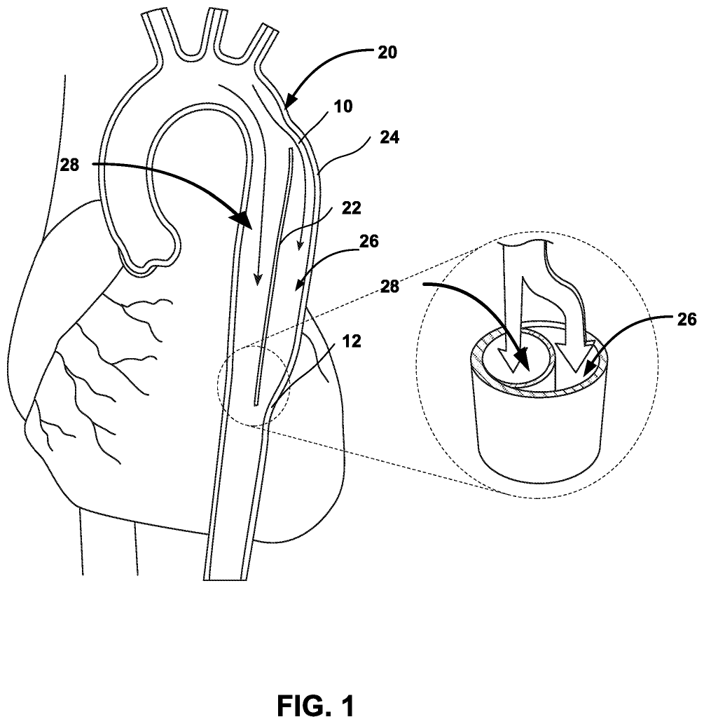

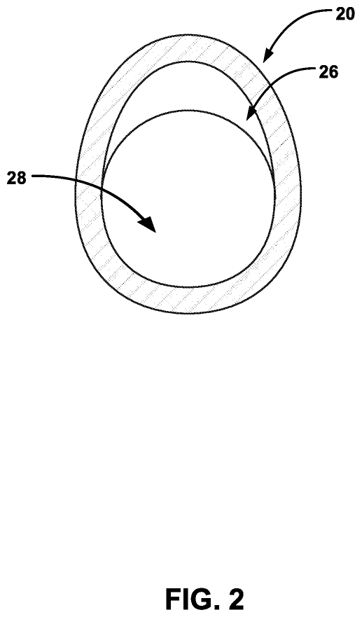

FIG. 1 illustrates a non-limiting example of a dissection in an aorta 20. A first tear 10 and a second tear 12 are formed in an inner layer 22 of the aorta 20 that separates or peels the inner layer 22 of the aorta 20 from the outer layer 24. Some blood flowing through the inner layer 22 goes through the first tear 10 and the second tear 12 (as depicted by arrows) in FIG. 1, thus creating a space between the inner layer 22 and the outer layer 24. The space created by the blood between the inner layer 22 and the outer layer 24 is referred to as the “false lumen” 26. The first tear 10 is referred as the primary entry point of the false lumen 26. The second tear 12 is referred as the “re-entry point” of the false lumen 26. The portion of the aorta 20 within the inner layer 22 of the aorta 20 along the dissection is referred to as the “true lumen” 28. The separated/peeled inner layer 22 is also termed as a dissection “flap” or a “septum”. As shown in FIG. 2, the cross section of the true lumen 28 is substantially circular and the cross section of the false lumen 26 is substantially crescent-shaped in nature.

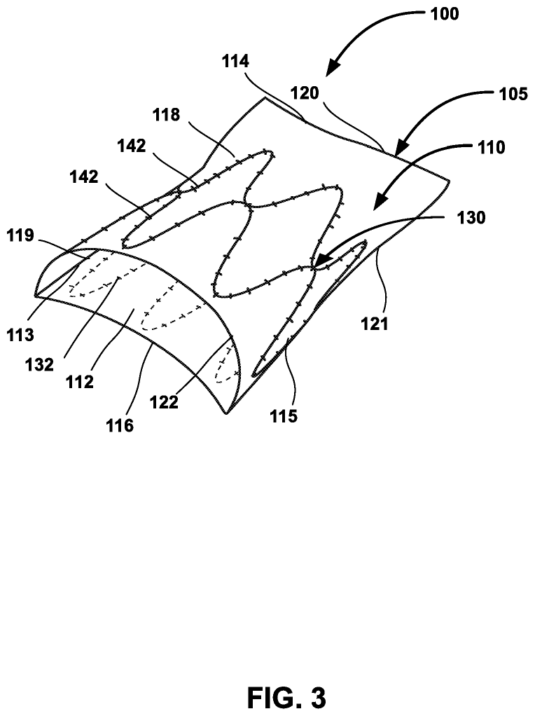

The various features and embodiments of a system and method for treatment of aortic dissection by using a stent graft of the present invention will now be described in conjunction with the accompanying figures, namely FIGS. 3-16.

FIGS. 3-5 illustrate a perspective view, a top view and front view of a stent graft or implant 100 respectively, according to an embodiment of the present invention. The stent graft 100 is configured to be deployed in the false lumen 26 (as seen in FIG. 1) to block blood flow from flowing into the false lumen 26 (as seen in FIG. 1) such that the stent graft 100 is effective in enabling complete thrombosis in the false lumen 26 (as seen in FIG. 1). The stent graft 100 comprises a graft structure 110 secured to a stent structure 130. The stent structure 130 serves to anchor the stent graft 100 in the false lumen 26 (FIG. 1) and provides structural support for the graft structure 110. Anchoring of the stent structure 130 in the false lumen 26 is achieved by the outward radial force exerted by the stent structure 130 which, in turn, is achieved by sizing the stent structure 130 or graft structure 110 larger than the false lumen size. In some other embodiment, the stent structure 110 can have integrally formed spikes or barbs that would help the stent graft 100 to anchor in the false lumen 26 and prevent it from getting dislodged from deployed position. The stent graft 100 comprises a closed top edge 105. The closed top edge 105, the graft structure 110 and the stent structure 130 of the stent graft 100 will now be described in detail in the below description.

The graft structure 110 comprises an inner graft 112 and an outer graft 118. The inner graft 112 has a first side edge 113, a top edge 114, a second side edge 115 and a bottom edge 116. The outer graft 118 has a first side edge 119, a top edge 120, a second side edge 121 and a bottom edge 122. The first side edge 113 of the inner graft 112 is connected to the first side edge 119 of the outer graft 118. The second side edge 115 of the inner graft 112 is connected to the second side edge 121 of the outer graft 118. The graft structure 110 is made up of an impermeable or low porosity material and the material could include but not limited to: polyethylene terephthalate (PET), polyurethane (PU), polytetrafluoroethylene (PTFE) and so on.

As seen in FIGS. 3-5, the stent structure 130 comprises multiple inner stents 132 and multiple outer stents 142 connected to the graft structure 110 such that a pair of inner stents 132 is connected to the inner graft 112 and a pair of outer stents 142 is connected to the outer graft 118. The inner graft 112 has a larger radius of curvature than the outer graft 118 and accordingly the inner stent 132 has a larger radius of curvature than the outer stent 142. As seen in FIGS. 3-5, the pair of inner stents 132 is positioned such that one stent of the pair of inner stents 132 is proximal or near to the top edge 114 and the remaining stent of the pair of inner stents 132 is distal or far from the top edge 114. Similarly, one stent of the pair of outer stents 142 is proximal or near to the top edge 120 and the remaining stent of the pair of outer stents 142 is distal or far from the top edge 120.

Each inner stent 132 as well as outer stent 142 has a preferably sinusoidal shape comprising a series of peaks and troughs wherein each inner stent 132 has total peaks P1 and the total troughs T1, wherein each outer stent 142 has total peaks P2 and the total troughs T2. As seen in FIGS. 3-5, the inner stent 132 is formed of sinusoidal shaped wires of a diameter D1 and the outer stent 142 is formed of sinusoidal shaped wires of a diameter D2. However, other shapes of inner stent 132 as well as outer stent 142 could be envisioned such as but not limited to: mesh shape, annular coil shape, flat shape, triangular shape, square shape, diamond shape and so on. The material for inner stent 132 as well as outer stent 142 could include but not limited to: stainless steel, nickel-titanium alloy commonly known as “Nitinol”, cobalt-chromium alloys, platinum, and tantalum alloys and so on.

Each inner stent 132 has a first end 133 and an opposite second end 134. Each outer stent 142 has a first end 143 and an opposite second end 144 (see FIG. 5). The first end 133 of each inner stent 132 is connected to the first end 143 of the outer stent 142 by any conventional methods/techniques including but not limited to: crimping, welding and so on. Similarly, the second end 134 of each inner stent 132 is connected to the second end 144 of the each outer stent 142 by any conventional methods/techniques including but not limited to: crimping, welding and so on.

The inner graft 112, the outer graft 118, the inner stents 132 and the outer stents 142 are thus connected to define the overall crescent shape of the stent graft 100. The crescent shape of the stent graft 100 is a result of different radius/diameter of curvature [corresponding to the arc] or arc length for the inner graft 112 and the outer graft 118. Preferably, the inner stents 132 have a larger radius of curvature than the outer stents 142. As seen in front view in FIG. 5, the stent graft 100 is substantially crescent shaped to functionally fit in the anatomy of the false lumen 26 (FIG. 1). Further, as seen in FIGS. 3-5, the top edge 114 of inner graft 112 and the top edge 120 of the outer graft 118 are joined together, thereby forming the closed top edge 105 of the stent graft 100. The stent graft 100 can be substantially flexible so as to conform to the curvature of the false lumen 26 (FIG. 1).

As seen in FIGS. 3-5, the pair of outer stents 142 is sutured together such that the peaks of one outer stent 142 are butted against the troughs of the remaining outer stent 142. Similarly, the pair of inner stents 132 is sutured together such that the peaks of one inner stent 132 are butted against the troughs of the remaining inner stent 132.

As further seen in FIGS. 3-5, the graft structure 110 is secured to the stent structure 130 by suturing. In other embodiments, the graft structure 110 is secured to the stent structure 130 by various techniques such as but not limited to: stapling, adhesive joining, clips, compression heat sealing, welding, heat encapsulation and so on.

In another embodiment (not shown in figures), the inner stent 132 and the outer stent 142 are substantially similar such that both the inner stent 132 and the outer stent 142 have same number of peaks and troughs as well as same wire diameter such that P1 is equal to P2, T1 is equal to T2 and D1 is equal to D2.

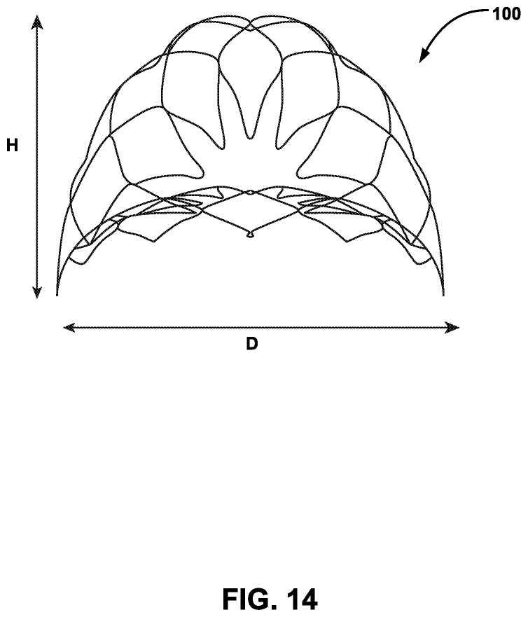

In another embodiment (not shown in figures), the inner stent 132 and the outer stent 142 may have a different number of peaks and troughs as well as different wire diameter. In an exemplary embodiment, the inner stent 132 has a larger number of peaks and troughs than the outer stent 142 such that P1 is larger than P2, and T1 is larger than T2. In an exemplary embodiment, the outer stent 142 is made from a wire diameter larger than the wire diameter of the inner stent 132 such that D2 is larger than D1. Further, each of the inner stent 132 and the outer stent 142 may have different base diameter and overall height as further described later in the description and seen in FIG. 14.

In another embodiment (not shown in figures), both outer stents 142 of the pair of outer stents 142 are integrally connected (formed) as a one-piece unit. The method/technique for integrally connecting the pair of outer stents 142 could include but not limited to: wire drawing, laser cutting, 3d printing and so on. Similarly, in another embodiment, both inner stents 132 of the pair of inner stents 132 are integrally connected as a one-piece unit. Additionally, in some embodiments, each of the outer stents 142 may be integrally connected with corresponding inner stents 132 to form a single continuous piece/unitary structure. Thus, as exemplified in FIG. 3, in total it would be two continuous sinusoidal stents (inner stent 132 or outer stent 142) placed adjacently and running all around the graft structure 110 covering inner graft 112 and outer graft 118. In some other embodiment, both or multiple outer stents 142 and both or multiple inner stents 132 may be integrally connected to form a single unitary stent structure 130.

In another embodiment (not shown in figures), the first side edge 113 of the inner graft 112 is connected and/or integrally joined/manufactured to the first side edge 119 of the outer graft 118 by various techniques such as but not limited to: suturing, stapling, adhesive joining, clips, compression heat sealing and so on. Similarly, the second side edge 115 of the inner graft 112 is connected and/or integrally joined/manufactured to the second side edge 121 of the outer graft 118 by various techniques such as but not limited to: suturing, stapling, adhesive joining, clips, compression heat sealing and so on.

In another embodiment (not shown in figures), the graft structure 110 is woven/fabricated such that the top edge 114 of inner graft 112 and the top edge 120 of the outer graft 118 are integrally joined/manufactured together, thereby forming the closed top edge 105 of the stent graft 100. Further, the first side edge 113 of the inner graft 112 is integrally joined/manufactured to the first side edge 119 of the outer graft 118 and the second side edge 115 of the inner graft 112 is integrally joined/manufactured to the second side edge 121 of the outer graft 118 respectively.

In another embodiment (not shown in figures), the closed top edge 105 of the stent graft 100 is formed by bringing the top edge 114 of inner graft 112 in close proximity with the top edge 120 of the outer graft 118, and then afterwards suturing/heat sealing the top edge 120 to the top edge 114, thereby forming the closed top edge 105 of the stent graft 100.

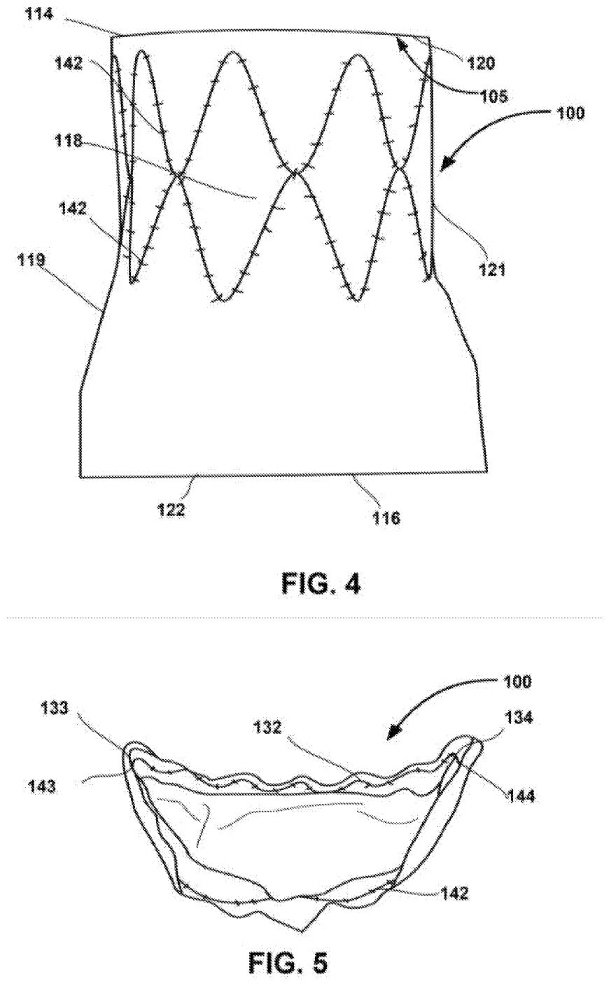

In another embodiment as seen in FIG. 6, the stent structure 130 comprises an inner stent 132 and an outer stent 142 connected to the graft structure 110 such that the inner stent 132 is connected to the inner graft 112 and the outer stent 142 is connected to the outer graft 118.

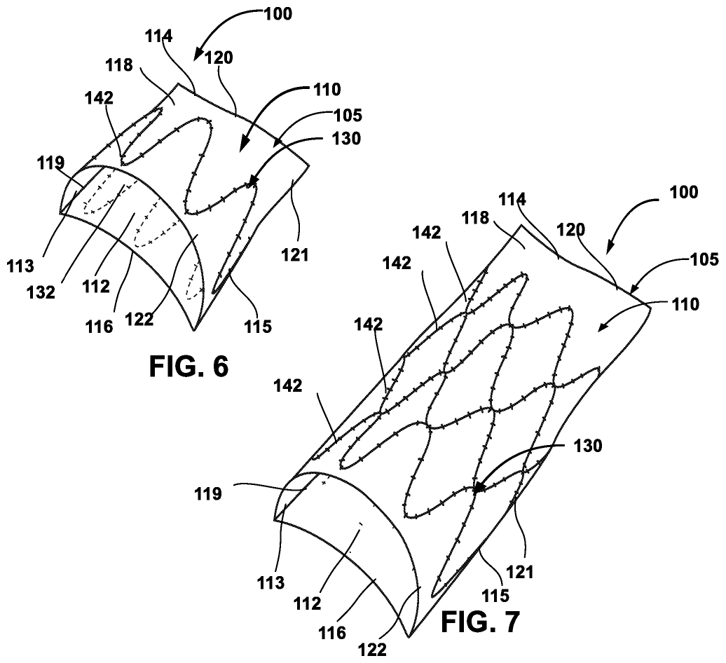

In another embodiment as seen in FIG. 7, the stent structure 130 comprises four inner stents 132 and four outer stents 142 connected to the graft structure 110 such that the four inner stents 132 are connected to the inner graft 112 and the four outer stents 142 are connected to the outer graft 118. The four outer stents 142 are sutured together such that the peaks of first outer stent 142 are butted against the troughs of the second outer stent 142, peaks of second outer stent 142 are butted against the troughs of the third outer stent 142 and so on. Similarly, the four inner stents 132 are sutured together such that the peaks of first inner stent 132 are butted against the troughs of the second inner stent 132, peaks of second inner stent 132 are butted against the troughs of the third inner stent 132 and so on.

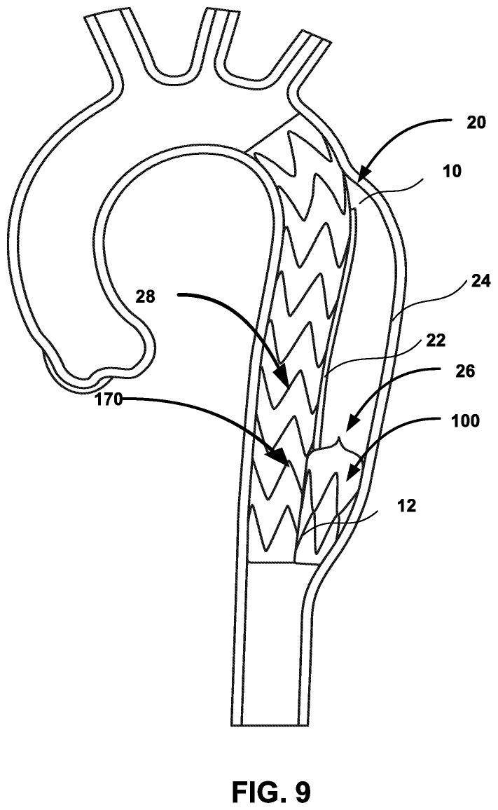

In an embodiment as seen in FIGS. 3-5, a pair of inner stents 132 is connected to the inner graft 112 and a pair of outer stents 142 is connected to the outer graft 118 respectively such that the pair of inner stents 132 and pair of outer stents 142 are positioned on the outside periphery of the inner graft 112 and outer graft 118 respectively. Thus, during deployment of stent graft 100 in the false lumen 26 (as seen in FIG. 9), at least a portion of inner stents 132 and outer stents 142 are in direct contact against the curvature of false lumen 26 (as seen in FIG. 9).

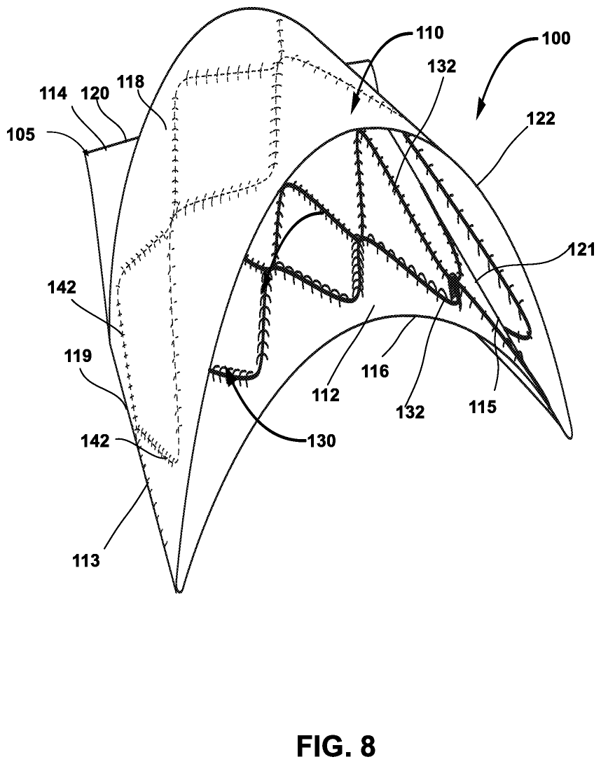

FIG. 8 illustrates a perspective view of a stent graft 100, according to another embodiment of the present invention. As seen in FIG. 8, the cross-sectional shape of the stent graft 100 is substantially crescent shaped to functionally fit in the anatomy of the false lumen 26. Further, as seen in FIG. 8, the top edge 114 of inner graft 112 and the top edge 120 of the outer graft 118 are joined together, thereby forming the closed top edge 105 of the stent graft 100 while the bottom edge 116 of inner graft 112 and the bottom edge 122 of the outer graft 118 are kept open, thus forming an open bottom edge of the stent graft 100.

As shown in FIG. 8, a pair of inner stents 132 is connected to the inner graft 112 and a pair of outer stents 142 is connected to the outer graft 118 respectively such that the pair of inner stents 132 and pair of outer stents 142 are positioned on the inside periphery of the inner graft 112 and outer graft 118 respectively. Thus, during deployment of stent graft 100 in the false lumen 26 (FIG. 9), the inner stents 132 and outer stents 142 are not in direct contact against the curvature of false lumen 26 (FIG. 9). Thus, at least some portion of inner graft 112 and outer graft 118 are in direct contact against the curvature of false lumen 26 (FIG. 9).

As seen in FIG. 9, the stent graft 100 of FIGS. 3-5 is shown in deployed position at re-entry point 12 of the false lumen 26 of aorta 20 such that the open bottom edge of the stent graft 100 allows blood flow through re-entry point 12 within the stent graft 100 wherein the blood flow is blocked from flowing from the stent graft 100 in the false lumen 26 due to the closed top edge 105 of the stent graft 100. Further as seen in FIG. 9, a true lumen stent graft 170 is deployed in the true lumen 28 wherein the true lumen stent graft 170 permits blood flow through the true lumen 28. The true lumen stent graft 170 is substantially tubular in shape and circular in cross section and is placed in the true lumen 28 to maintain patency of the true lumen 28.

In an embodiment, the true lumen stent graft 170 is configured such that it blocks blood flow from flowing in the false lumen 26 through the primary entry point 10. Further, in another embodiment, multiple true lumen stent grafts 170 are deployed in the true lumen 28. In the case of multiple true lumen stent grafts 170, the true lumen stent grafts 170 can be positioned adjacent to one another, spaced apart or can be positioned relative to one another so as to at least partially overlap. The true lumen stent grafts 170 can be flexible so as to conform to the curvature of the aorta. Some embodiments of the true lumen stent grafts 170 can have large spaces between the struts of stent to allow for flow into branch vessels of the aorta.

As seen in FIG. 9, the stent graft 100 is deployed at the re-entry point 12 of the false lumen 26 in the type B dissection. Further, it is obvious to the one skilled in the art that the stent graft 100 could also be deployed in the type A dissection with little or no variation to block blood flow from flowing though false lumen 26. Further, it should also obvious to the one skilled in the art that the stent graft 100 could also be deployed near the re-entry point 12 (in proximity to the re-entry point 12) with little or no variation to block blood flow from flowing though false lumen 26. For instance, the stent graft 100 could also be deployed midway in the false lumen 26.

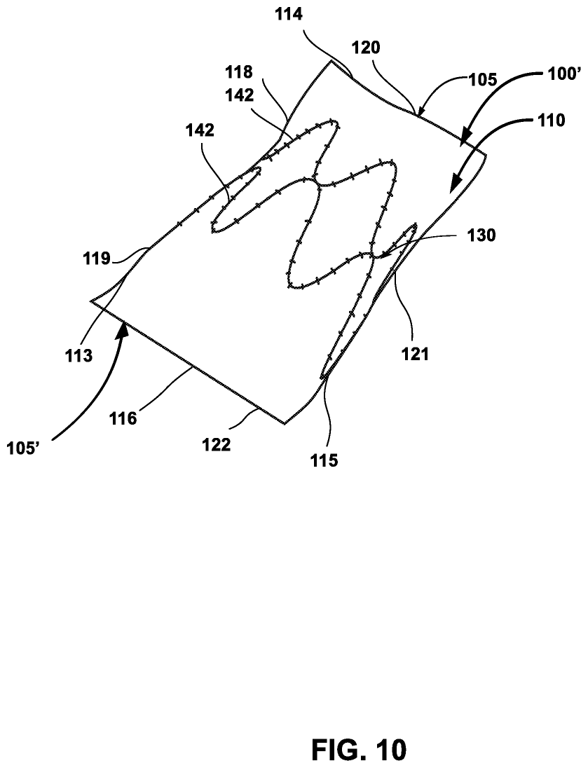

In another embodiment as shown in FIG. 10, a stent graft 100′ has a closed top edge 105 as well as a closed bottom edge 105′. The stent graft 100′ is similar to the stent graft 100 shown in FIGS. 3-5 except for modifications associated with the bottom edge of the stent graft 100. As seen in FIG. 10, the bottom edge 116 of inner graft 112 and the bottom edge 122 of the outer graft 118 are joined together, thereby forming a closed bottom edge 105′ of the stent graft 100′. Also, the top edge 114 of inner graft 112 and the top edge 120 of the outer graft 118 are joined together, thereby forming the closed top edge 105 of the stent graft 100′, similar to closed top edge 105 of the stent graft 100 as shown in FIGS. 3-5.

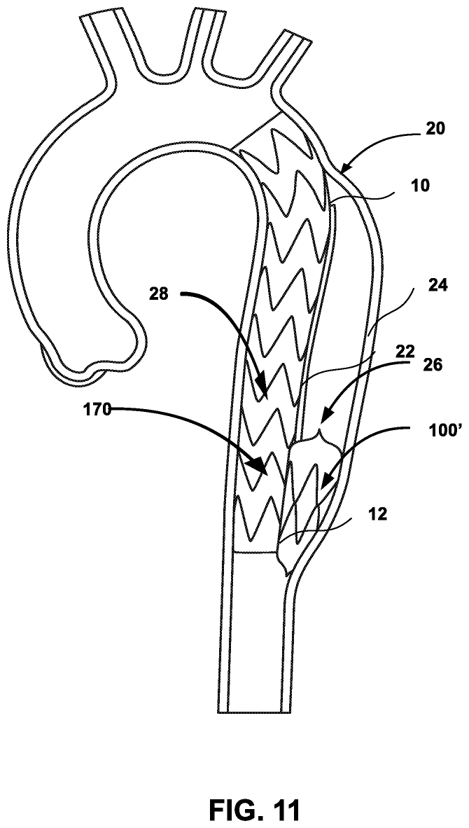

As seen in FIG. 11, the stent graft 100′ is shown in deployed position at re-entry point 12 of the false lumen 26 of aorta 20 such that the closed bottom edge (not shown in figures) of the stent graft 100′ blocks blood flow from flowing from the re-entry point 12 within the stent graft 100′. Further as seen in FIG. 11, a true lumen stent graft 170 is deployed in the true lumen 28 wherein the true lumen stent graft 170 permits blood flow through the true lumen 28. The true lumen stent graft 170 is placed in the true lumen 28 to maintain patency of the true lumen 28. Further, in another embodiment, multiple true lumen stent grafts 170 are deployed in the true lumen 28. In the case of multiple true lumen stent grafts 170, the true lumen stent grafts 170 can be positioned adjacent to one another, spaced apart or can be positioned relative to one another so as to at least partially overlap. The true lumen stent grafts 170 can be flexible so as to conform to the curvature of the true lumen 28. Some embodiments of the true lumen stent grafts 170 can have large spaces between the struts to allow for flow into branch vessels of the aorta.

In an embodiment, the graft structure 110 is woven/fabricated such that the bottom edge 116 of inner graft 112 and the bottom edge 122 of the outer graft 118 are integrally joined together, thereby forming the closed bottom edge (not shown in figures) of the stent graft 100′.

In another embodiment, the closed bottom edge (not shown in figures) of the stent graft 100′ is formed by bringing the bottom edge 116 of inner graft 112 in close proximity with the bottom edge 122 of the outer graft 118, and then afterwards suturing/heat sealing the bottom edge 122 to the bottom edge 116, thereby closing the bottom edge of the stent graft 100′.

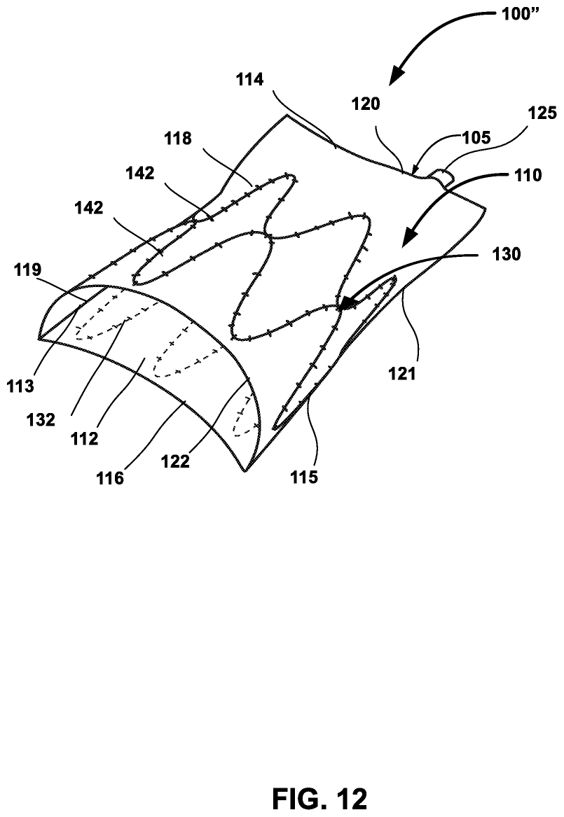

FIG. 12 illustrates a perspective view of a stent graft 100″, according to another embodiment of the present invention. The stent graft 100″ is similar to the stent graft 100 shown in FIGS. 3-5 except for modifications associated with the top edge 105 of the stent graft 100. As seen in FIG. 12, the top edge 114 of inner graft 112 and the top edge 120 of the outer graft 118 are joined together at an intersection point, wherein at least one hole 125 is formed in the intersection point. The hole(s) 125 is provided at the closed top edge 105 of the stent graft 100″ to allow blood perfusion for a limited time until collateral flow is developed to support the vessels connected to the false lumen 26 (FIG. 9). The hole(s) 125 is configured such that after sufficient time, the hole(s) 125 is automatically closed (filled) or by closed by deploying another stent graft (without any hole 125) during a separate clinical intervention within the stent grant having hole 125 such that blood perfusion will not take place from the hole(s) 125 after sufficient time. Further, the hole(s) 125 may also be configured at both the closed top edge 105 and the closed bottom edge 105′ of the stent graft 100′ shown in FIG. 11.

In an embodiment (not shown in figures), the hole(s) 125 is integrally formed at the closed top edge 105 of the stent graft 100″ or at the closed top and bottom edges 105,105′ during manufacturing of the stent graft 100″ and stent graft 100′ respectively using a suitable technique including but not limited to drilling, punching, suturing and so on.

In another embodiment (not shown in figures), the hole(s) 125 is made of a material such that after sufficient and/or predetermined time, the hole(s) 125 is automatically closed such that blood perfusion will not take place from the hole(s) 125 after sufficient and/or predetermined time.

In another embodiment (not shown in figures), the hole(s) 125 has constructional design features such that after sufficient time, the hole(s) 125 is automatically closed such that blood perfusion will not take place from the hole(s) 125 after sufficient time.

In another embodiment, the hole(s) 125 is formed in situ during/after deployment of the stent graft 100″ in the false lumen 26 using a balloon catheter 150 having a balloon 160, and an electrode running across the catheter tube up to the tip, which will be described in detail in below description.

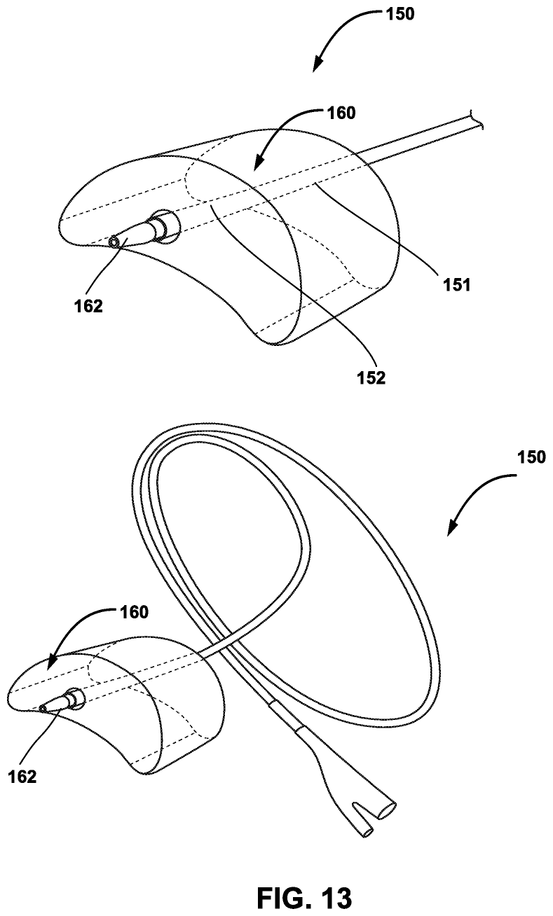

FIG. 13 illustrates a perspective view of a balloon catheter 150 with a crescent-shaped balloon 160 attached to catheter tube 151. The balloon 160 enables the stent graft 100,100′,100″ to functionally fit better in the anatomy of the false lumen, according to an embodiment of the present invention. In an embodiment, the stent graft 100,100′, 100″ may be deployed using some conventional catheter tube (not seen). The conventional catheter tube will have the proposed graft stent loaded therein in a crimped form wrapped eccentrically about the inner tube/guide wire lumen passing through the catheter tube. The conventional catheter tube can be used for endovascularly deploying a stent graft 100, 100′, 100″ in the false lumen 26 (as shown in FIG. 9 and FIG. 11) through a body opening or a body cut performed during surgery preferably via transfemoral access while using established image guidance such as X-ray fluoroscopy, intravascular ultrasound (IVUS) and so on. The deployment of the stent graft 100,100′,100″ is preferably monitored by including one or more radiopaque markers on the top edges 114, 120 and/or the bottom edges 116, 122 of the outer graft 118 and inner graft 112 or anywhere appropriate on the surface of the outer graft 118 and inner graft 112. Afterwards, the stent graft 100, 100′, 100″ which is initially in the crimped state is delivered and deployed by the catheter in the false lumen 26. After deployment, the catheter is removed from the patient body. In an embodiment, the balloon catheter 150 with balloon 160 may be used for deploying the stent graft. During deployment, the stent graft 100,100′,100″ is inflated at a desired pressure to deploy the stent graft 100, 100′, 100″ in the false lumen 26 (as shown in FIG. 9 and FIG. 11) at the re-entry point 12, resulting in expansion of stent structure 130 and/or graft structure 110, thereby engaging the stent graft 100, 100′, 100″ against the curvature of the false lumen 26 (as shown in FIG. 9 and FIG. 11). Afterwards, the balloon 160 of the balloon catheter 150 is deflated and the catheter 150 is removed from the patient body. The balloon 160 is made of a non-compliant or semi-compliant material such as but not limited to: nylon, PET and so on.

Further, in an embodiment, a crescent-shaped balloon 160 is deployed using the catheter 150. The balloon 160 is inflatable and initially introduced in deflated state. The balloon 160 is later inflated to desired pressure or desired volume and is made to contact with the deployed stent graft (100, 100′, 100″) thereby enabling the stent graft (100, 100′, 100″) to functionally fit better in the anatomy of the false lumen 26. Afterwards, the balloon is deflated and the catheter 150 is removed from the patient body. The balloon 160 is made of a flexible/compliant or semi-compliant material such as but not limited to: polyurethane (PU), latex, and so on.

In another embodiment (not shown in figures), the crescent-shaped balloon 160 could also function as an inflatable tip (not shown in figures) such that the crescent-shaped balloon 160 deploys the stent graft (100, 100′, 100″) which is initially in the substantially deflated position in the false lumen 26 (as shown in FIG. 9 and FIG. 11) at the re-entry point 12 as well as enabling the deployed stent graft (100, 100′, 100″) to functionally fit better in the anatomy of the false lumen 26 (as shown in FIG. 9 and FIG. 11).

In an embodiment as seen in FIG. 13, the balloon 160 has a cutting arrangement 162 at the tip of the balloon 160 to make the hole(s) 125 at the closed top edge 105 of the stent graft 100″. The cutting arrangement 162 can include but not limited to: cutting tip, heating electrode, radiofrequency ablation electrode, cryo ablation electrode and so on. The cutting arrangement 162 at the tip of the balloon 160 is configured to cut a hole(s) 125 in the closed top edge 105 of the stent graft 100″ during/after deployment of the stent graft 100″ in situ in the false lumen 26 (as shown in FIG. 9 and FIG. 11) by a catheter 150 and/or balloon 160.

FIG. 14 illustrates a stent graft (100, 100′, 100″) having a base diameter D and an overall height H. Depending on the dimensions of the false lumen 26 (as shown in FIG. 9 and FIG. 11) and requirement of the patient, the base diameter D of the stent graft (100, 100′, 100″) could vary from 10 millimeters to 100 millimeters, preferably in the range of 20 millimeters to 60 millimeters. Further, the overall height H of the stent graft 100 could vary from 5 millimeters to 80 millimeters, preferably in the range of 10 millimeters to 60 millimeters. Further, each of the inner stent 132 and the outer stent 142 may have different base diameter D and overall height H. Further, it should be obvious that the size of the stent graft (100, 100′, 100″) is not to be considered a limiting embodiment of the present invention.

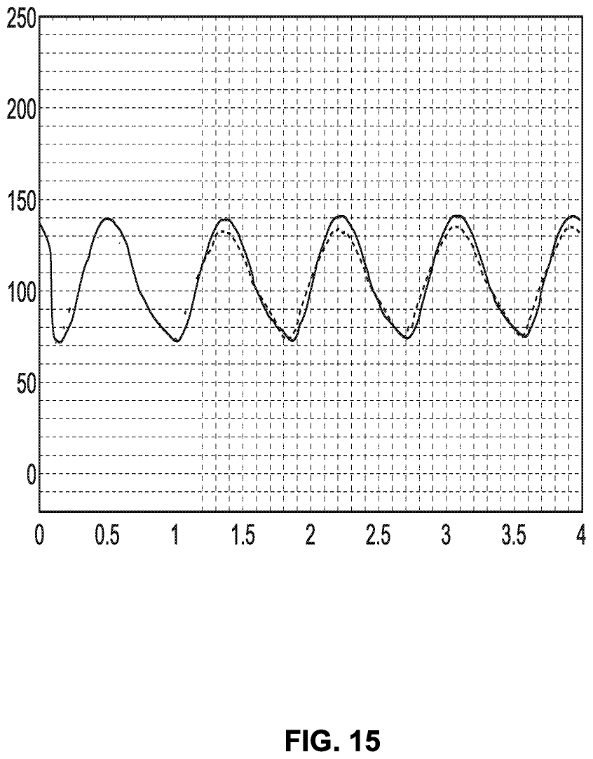

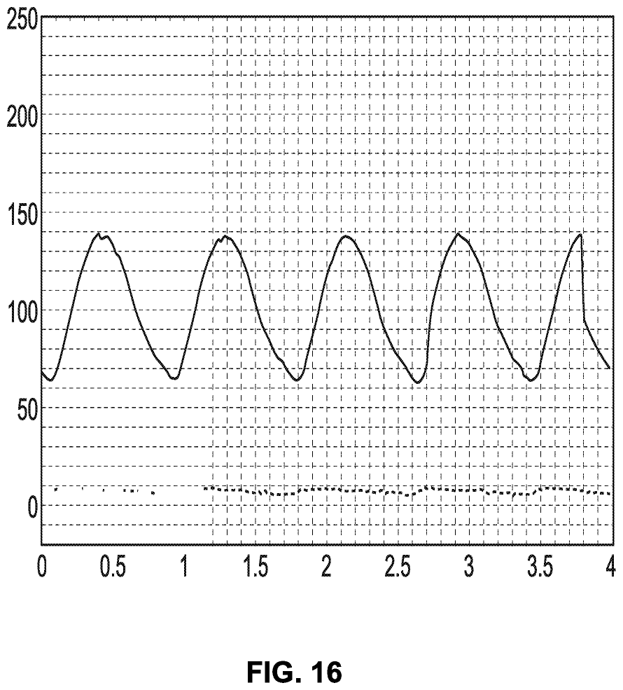

FIG. 15 and FIG. 16 shows pressure versus time graphs in the false lumen 26 without the stent graft (100, 100′, 100″) in the false lumen 26 and with the stent graft (100, 100′, 100″) deployed in the false lumen 26 (as shown in FIG. 9 and FIG. 11) respectively. A pressure measurement device (not shown in figures) is installed in a simulated in vitro test setup simulating the false lumen 26 and the pressure readings (in mmHg) are displayed on a computing device such as but not limited to: smartphone, computer, laptop, PDA and so on. The X-axis denotes the time (in seconds/minutes) while the Y-axis denotes the pressure (in mmHg) in the simulated aortic dissection in vitro test setup. The pressure readings of true lumen 28 (as shown in FIG. 9 and FIG. 11) is represented by solid lines and the pressure readings of false lumen 26 (as shown in FIG. 9 and FIG. 11) is represented by dotted lines. The pressure readings observed in the aortic dissection follows sinusoidal profile due to the pulsatile nature of physiological blood flow. Thus, the average/mean pressure in true lumen 28 is considered as average of maximum/systolic pressure and minimum/diastolic pressure in true lumen 28. Similarly, the average/mean pressure in false lumen 26 is considered as average of maximum/systolic pressure and minimum/diastolic pressure in false lumen 26. The pressure readings without the stent graft (100, 100′, 100″) versus with the stent graft (100, 100′, 100″) deployed is compared to determine efficacy of stent graft (100, 100′, 100″).

As seen in FIG. 15, the pressure difference between the false lumen 26 (as shown in FIG. 9 and FIG. 11) and the true lumen 28 (as shown in FIG. 9 and FIG. 11) is very negligible. This suggests that when there is no stent graft (100, 100′, 100″) in the false lumen 26, the blood flow through the false lumen 26 continues to pressurize the false lumen which is a serious medical condition as previously described.

As seen in FIG. 16, when the stent graft (100, 100′, 100″) is deployed in the false lumen 26 (as shown in FIG. 9 and FIG. 11), the pressure in the false lumen 26 (as shown in FIG. 9 and FIG. 11) is very low (approximately 10-15 mmHg) which suggests almost complete occlusion (no blood flow through false lumen) of false lumen 26 (as shown in FIG. 9 and FIG. 11) by deploying the stent graft (100, 100′, 100″) in the false lumen 26 (as shown in FIG. 9 and FIG. 11). Further, there is clinical evidence that suggests that such near complete occlusion of the false lumen 26 (as shown in FIG. 9 and FIG. 11) could lead to complete thrombosis of the false lumen 26 (as shown in FIG. 9 and FIG. 11), which can further assist in treating the effective treatment of aortic dissection.

According to some embodiment, the stent graft (100,100′,100″) may have an integrated sealing ring (not seen) either on the top edge 114 or bottom edge 116 or both top and bottom edges 114,116 of the inner and outer grafts 112,118 to achieve better conformability and therefore sealing against the wall of the false lumen. The sealing ring may have a circular, annular or sinusoidal shape and comprise of an expansible material such as foam, hydrogels and so on, which allow for crimping of the stent graft (100,100′ 100″) in a catheter tube and expand after deployment.

An exemplary method of treating an aortic dissection will now be described with reference to FIGS. 1-14.

Firstly, an aortic dissection is located in the aorta 20 using any conventional techniques known in the art. Afterwards, a true lumen stent graft 170 is deployed in a true lumen 28 of an aortic dissection of the aorta 20. Afterwards, a stent graft (100, 100′, 100″) is introduced via re-entry point 12 in the false lumen 26 of an aortic dissection such that the stent graft (100, 100′, 100″) is deployed at or in the proximity of the re-entry point 12 in the false lumen 26. The geometry and the constructional features of the stent graft (100, 100′, 100″) blocks blood flow from flowing through the false lumen 26. Wherein the true lumen stent graft 170 is configured to allow blood flow from flowing through the true lumen 28.

The system and method for treatment of aortic dissection involving use of a stent graft (100, 100′, 100″) of the present invention including various components, parts thereof may be configured in many different shapes, sizes and using different kinds of materials, including but not limited to metals, plastics, ceramics, composites, polymers, rubber, silicone and one should not construed these aspects to be a limiting factor for the invention disclosed herein.

It should be understood that the various parts of the various embodiments of stent graft (100, 100′, 100″) of the present invention are similar and interchangeable. It is obvious to the one skilled in the art that the various parts of stent graft (100, 100′, 100″) of one embodiment of the present invention could be considered for other embodiments with little or no variation.

It should be understood according to the preceding description of the present invention that the same is susceptible to changes, modifications and adaptations, and that the said changes, modifications and adaptations fall within scope of the appended claims.

1. A stent graft (100, 100′, 100″), comprising:
a graft structure (HO) comprising an inner graft (112) and an outer graft (118); wherein the inner graft (112) has a top edge (114) and a bottom edge (116); and the outer graft (118) has a top edge (120) and a bottom edge (122);
a stent structure (130) comprising at least one inner stent (132) and at least one outer stent (142) connected to the inner graft (112) and the outer graft (118) respectively; and
wherein, the inner graft (112) has a larger radius of curvature than the outer graft (118).

2. The stent graft (100, 100′, 100″) of claim 1, wherein the top edge (114) of the inner graft (112) and the top edge (120) of the outer graft (118) are joined together to form a closed top edge (105) of the stent graft (100,100″).

3. The stent graft (100, 100′, 100″) of claim 1, wherein the bottom edge (116) of the inner graft (112) and the bottom edge (122) of the outer graft (118) are joined together to form a closed bottom edge (105′) of the stent graft (100′).

4. The stent graft (100, 100′, 100″) of claim 1, wherein the stent graft (100, 100′, 100″) is configured for deployment at a re-entry point (12) of a false lumen (26) of an aortic dissection.

5. The stent graft (100, 100′, 100″) of claim 1, wherein the at least one inner stent (132) and the at least one outer stem (142) connected to the inner graft (112) and the outer graft (118) respectively to define an overall crescent shape of the stent graft (100, 100′, 100″).

6. The stent graft (100,100′, 100″) of claim 5, wherein the crescent shape of the stent graft (100, 100′,100″) is a result of varying base diameter and overall height for the inner graft (112) and the outer graft (118) and varying base diameter and overall height for the at least one inner stent (132) and the at least one outer stent (142).

7. The stent graft (100, 100′, 100″) of claim 1, further comprising at least one hole (125) provided at the closed top edge (105) of the stent graft (100,100′) and/or the closed bottom edge (105′) of the stent graft (100′).

8. The stent graft (100, 100′,100″) of claim 1, wherein the stent structure (130) serves to anchor the stent graft 100 in the false lumen (26) and provides structural support for the graft structure (110).

9. The stent graft (100, 100′, 100″) of claim 8, wherein the stent structure (130) or the graft structure (110) is sized larger than the false lumen (26) to support the anchoring of the stent structure (130)) in the false lumen (26).

10. The stent graft (100, 100′, 100″) of claim 8, wherein the stent structure (110) comprises one or more integrally formed spikes or barbs that help the stent graft (100, 100′, 100″) to anchor in the false lumen (26) and prevent it from getting dislocated from deployed position.

11. The stent graft (100, 100′,100″) of claim 1 further comprising one or more radiopaque markers located at least on the top edges (114, 120) and the bottom edges (116, 122) of the outer graft (118) and inner graft (112) or at some other location on the surface of the outer graft (118) and inner graft (112).

12. The stent graft (100, 100′, 100″) of claim 1, wherein the at least one inner stent (132) and the at least one outer stent (142) are substantially similar having same number of peaks and troughs as well as same wire diameter, same base diameter, and overall height.

13. The stent graft (100, 100′, 100″) of claim 1, wherein the inner stent (132) and the outer stent (142) are substantially different having a different number of peaks and troughs as well as different wire diameter, different base diameter, and overall height.

14. The stent graft (100) of claim 1, comprising at least the top edges (114, 120) or the bottom edges (116, 122) of the inner graft (112) and outer graft (118) comprising a sealing ring having a circular, annular or sinusoidal shape and comprising of an expansive material.

15. A system, comprising:
a stent graft (100, 100′,100″) for deploying in a false lumen (26) to block the blood flow therethrough, the stent graft comprising:
a graft structure (110) comprising an inner graft (112) and an outer graft (118); wherein the inner graft (112) has a top edge (114) and a bottom edge (116); and the outer graft (118) has a top edge (120) and a bottom edge (122);
a stent structure (130) comprising at least one inner stent (132) and at least one outer stent (142) connected to the inner graft (112) and the outer graft (118) respectively;
wherein the inner graft (112) has a larger radius of curvature than the outer graft (118); and
a true lumen stent graft (170) for deploying in a true lumen (28) to allow blood to flow therethrough, wherein the true lumen stent graft (170) is substantially tubular in shape and circular in cross section.

16. The system of claim 15 further comprising a balloon catheter (150) having a balloon (160) for enabling the stent graft (100, 100′,100″) to functionally fit better in the anatomy of a false lumen (26) formed as a result of aortic dissection.

17. (canceled)

18. (canceled)

19. (canceled)

20. (canceled)

21. (canceled)

22. (canceled)

23. The system of claim 15 further comprising at least one hole (125) provided at the closed top edge (105) of the stent graft (100,100′) and/or the closed bottom edge (105′) of the stent graft (100′).

24. (canceled)

25. (canceled)

26. The system of claim 15 further comprising one or more radiopaque markers located at least on the top edges (114, 120) and the bottom edges (116, 122) of the outer graft (118) and inner graft (112) or at some other location on the surface of the outer graft (118) and inner graft (112).

27. The system of claim 16, wherein the balloon (160) having a cutting arrangement (162) located at a tip of the balloon (160) to make the hole (125) at the closed top edge (105) and/or the closed bottom edge (105′) of the stent graft (100,100′).

28. The system of claim 15, wherein at least the top edges (114, 120) or the bottom edges (116, 122) of the inner graft (112) and outer graft (118) comprising a sealing ring having a circular, annular or sinusoidal shape and comprising of an expandable material.

29. (canceled)

30. (canceled)

31. (canceled)
